(19) United States
(12) Patent Application Publication
(10) Pub. No.: US 2025/0302391 A1
(43) Pub. Date: Oct. 2, 2025

(54) METHOD FOR ACQUIRING A MAGNETIC RESONANCE IMAGE DATASET OF A BODY PART OF A SUBJECT

(71) Applicant: Siemens Healthineers AG, Forchheim (DE)

(72) Inventors: Daniel Nicolas Splitthoff, Uttenreuth (DE); Julian Hossbach, Erlangen (DE); Bryan Clifford, Belmont, MA (US); Wei-Ching Lo, Charlestown, MA (US); Stephen Farman Cauley, Winchester, MA (US); Daniel Polak, Erlangen (DE)

(21) Appl. No.: 19/094,363

(22) Filed: Mar. 28, 2025

(30) Foreign Application Priority Data
Mar. 28, 2024 (EP) 24167445

(51) Int. Cl. A61B 5/00; A61B 5/055; G01R 33/56; G01R 33/565

(52) U.S. Cl. CPC A61B 5/7264; A61B 5/055; G01R 33/5608; G01R 33/56509; A61B 2560/0238

(57) ABSTRACT

A method for acquiring a magnetic resonance image dataset of a body part of a subject includes acquiring a low-resolution magnetic resonance image of the body part, optionally acquiring a reference set of additional k-space lines within a central region of k-space, and acquiring further sets of additional k-space lines within a central region of k-space at intervals throughout the imaging protocol. The method includes applying a trained machine learning model to a dataset including the low-resolution image in k-space notation and a further set of additional k-space lines. A set of pose parameters is generated. The pose parameters are used for prospective and/or retrospective motion correction of the magnetic resonance image dataset.

This application claims the benefit of European Patent Application No. EP 24167445.6, filed on Mar. 28, 2024, which is hereby incorporated by reference in its entirety.

### BACKGROUND

The present embodiments relate to a method for acquiring a magnetic resonance image dataset of a body part of a subject, a computer-implemented method for providing a trained machine learning model, a computer program, and a magnetic resonance imaging apparatus.

Independent of the grammatical term usage, individuals with male, female, or other gender identities are included within the term.

Patient motion is one of the most common and costly type of artifacts in Magnetic Resonance Imaging (MRI) and may seriously degrade the diagnostic quality of magnetic resonance (MR) examinations.

Motion correction may be done retrospectively (e.g., after the acquisition of the magnetic resonance image dataset) or prospectively (e.g., during the acquisition). Both approaches try to minimize the effect of motion and mitigate motion artifacts, but often lack the applicability for the clinical routine.

Retrospective methods correct for motion artefacts after the data acquisition in the course of the image reconstruction. By including motion parameters into the MR forward model, these techniques account for the patient's motion in the final image reconstruction and therefore reduce motion artefacts. In some techniques, the motion data may be derived from the acquired k-space data itself. For multi-shot acquisitions, the goal in retrospective motion correction techniques is to extract the per shot motion parameters and the motion-free image simultaneously. This may be accomplished by either minimizing an image quality metric, such as image entropy, or by minimizing the data consistency error of a so-called “SENSE+motion” forward model, as described, for example, in L. Cordero-Grande, R. Teixeira, E. Hughes, J. Hutter, A. Price, Hajnal, “Sensitivity Encoding for Aligned Multishot Magnetic Resonance Reconstruction,” IEEE Trans. Comput. Imaging, vol. 2, no. 3, pp. 266-280, 2016.

In “Scout accelerated motion estimation and reduction (SAMER),” Magn. Reson. Med., vol. 87, pp. 163-178, 2022, https://doi.org/10.1002/mrm.28971, D. Polak, D. N. Splitthoff, B. Clifford, W.-C. Lo, S. Huang, J. Conklin, L. L. Wald, K. Setsompop, and S. Cauley propose a technique that utilizes a single rapid scout scan to drastically reduce the computational cost of motion estimation. The scout image contains center of k-space information that is compared against the k-space data of the actual MR acquisition for each shot, to derive the subject's motion. This strategy is used to completely avoid the alternating optimization of subject motion parameters and image volume, which is otherwise required in other data-consistency based retrospective motion correction techniques. In the SAMER-technique, a motion trajectory of the subject is first estimated, and the motion trajectory is then used in a motion-aware parallel image reconstruction, using, for example, a “SENSE+motion” forward model to yield the motion-mitigated image. In D. Polak, J. Hossbach, D. N. Splitthoff et al., “Motion guidance lines for robust data consistency-based retrospective motion correction in 2D and 3D MRI,” Magn. Reson. Med. 2023:1-14, doi: 10.1002/mrm.29534, D. Polak et al. have extended the SAMER method to include the repeated acquisition of a small number of motion guidance lines in each shot, which are used for motion estimation by being compared with the data from the scout scan. This allows an estimation of motion parameters shot-by-shot.

Retrospective motion correction techniques often require a significant processing time and thereby may increase the reconstruction time to a level that is unacceptable in clinical routine. Further, rotation and spin-history artifacts are not always fully correctable retrospectively.

Prospective motion correction requires an accurate tracking of the subject's motion. This may be done by additional hardware to track patient motion during the MR examination (e.g., cameras) or separate navigator scans. Camera-based approaches may lack the accuracy to differentiate skin motion from rigid body motion (e.g., of the head). Navigators require thorough sequence changes potentially prolonging the acquisition, as explained in Maclaren, J., Herbst, M., Speck, O. et al., “Prospective motion correction in brain imaging: A review,” Magn Reson Med, 2013; 69:621-636.

Recently, deep learning techniques have increasingly been applied to image reconstruction in MRI. In Hossbach J, Splitthoff D, Cauley S, et al., “Deep Learning-Based Motion Quantification from k-space for Fast Model-Based MRI Motion Correction,” Med. Phys., 2023; 50:2148-2161, it has been demonstrated that it is possible to use a neural network to estimate subject motion between two acquisitions. However, the method requires the use of special sequence features, such as echo trains that span over the full extent of k-space (e.g., each echo train has data in the low-resolution part of k-space). The method cannot be universally applied to any imaging sequence.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the above-mentioned problems of prior art motion techniques are alleviated. For example, a method for acquiring a motion corrected MRI dataset of a body part of a subject, in which motion correction does not delay acquisition or reconstruction of the image data, that works with any type of imaging sequence and provides a good motion correction performance is provided.

According to a first aspect, the present embodiments are directed to a method for acquiring a magnetic resonance image dataset of a body part of a subject. The magnetic resonance image dataset is acquired using an imaging protocol. Spatial encoding is performed using phase encoding gradients along at least one phase encoding direction and frequency encoding gradients along a frequency encoding direction. k-space is sampled during the imaging protocol by acquiring a plurality of imaging k-space lines oriented along the frequency encoding direction and having different positions in the at least one phase encoding direction. The method includes the acts of: (a) acquiring or providing a low-resolution magnetic resonance image of the body part; (b1) optionally acquiring a reference set of additional k-space lines within a central region of k-space (e.g., at the beginning of the imaging protocol); (b2) acquiring further sets of additional k-space lines within a central region of k-space at intervals throughout the imaging protocol; and (c) applying a trained machine learning model to a dataset including the low-resolution image in k-space notation and a further set of additional k-space lines. A set of pose parameters is generated. The pose parameters are an estimation of the pose of the subject during the acquisition time of the further set of additional k-space lines.

The present embodiments use a trained machine learning model (e.g., a neural network (NN)) to estimate pose parameters from the additional k-space lines that are acquired in addition to the imaging protocol in a similar fashion as in the SAMER technique. The set of additional k-space lines is acquired at intervals throughout the imaging protocol, and those additional k-space lines, also referred to as guidance lines (GL), are used to estimate pose parameters, referred to herein as motion parameters. The estimated pose parameters provide a sufficiently good estimate of the varying position of the imaged body part throughout the imaging protocol. A time sequence of pose parameters may also be referred to as motion trajectory.

In the above-described SAMER technique, such a motion trajectory is used in the image reconstruction in order to allow retrospective motion correction. However, the SAMER technique has the disadvantage that the pose parameters are estimated by the registration of the additionally acquired guidance lines to a previously acquired scout image. This motion estimation is performed by numerically solving an optimization problem that requires as input processed data such as coil sensitivities and the reconstructed scout, leading to a processing time that may be too long to instantly allow use of the estimated pose parameters for prospective motion correction (e.g., right from the start of the sequence while the imaging protocol is being performed). Rather, the SAMER technique may be limited to retrospective motion correction. The present embodiments provide a significant improvement over the SAMER method by applying a trained machine learning model to a dataset including the guidance lines and a low-resolution image of the body part, where the output of the trained machine learning model is a set of pose parameters. This act in k-space is significantly faster than the previously used numerical optimization, in which each set of pose parameters was determined independently by minimizing the data consistently error of a forward model (e.g., a SENSE+motion forward model) that is computationally more demanding, since it involves both image and k-space operations. The trained machine learning model may operate in k-space, so that both the low-resolution image and the guidance lines are input in k-space notation. This has the additional advantage that no scout image reconstruction is required and the pre-processing of the dataset, to which the trained machine learning model is applied, may be kept to a minimum. For example, no coil sensitivity maps calculation is required. This further reduces the processing time required for the estimation of the pose parameters. Overall, the time required for estimating pose parameters from a set of additional k-space lines (e.g., guidance lines) is so small (e.g., less than 15 ms or less than 8 ms) that the pose parameters may be used for prospective motion correction of the magnetic resonance image dataset. For example, the pose parameters may be used to adapt a field of view of the MRI dataset during the imaging protocol. In other words, if the patient has moved during the acquisition of an MRI image dataset, this is reflected in the next set of pose parameters, and the acquisition parameters may be immediately adapted to the new position for the rest of the acquisition. This prospective motion correction provides better results than retrospective motion correction, because the prospective motion correction avoids gaps in k-space in the acquired data, which may otherwise occur, and which are not fully correctable retrospectively.

In addition, the use of a trained machine learning model for the estimation of posed parameters is advantageous over the previously used numerical optimization. For example, the use of a trained machine learning model has proven to work even if the low-resolution image and the additional k-space lines, to which the trained machine learning model is applied, have different contrasts. Different contrasts may arise if the low-resolution image and the guidance lines have been acquired, for example, at different echo times and/or using different magnetization preparation and/or different repetition times (e.g., different sequence parameters or a different type of imaging sequence). In the SAMER technique, the scout image used in the estimation of the motion trajectory is specifically acquired for this purpose and will therefore have the same contrast as the motion guidance lines acquired throughout the imaging protocol. This necessarily requires a dedicated scout image and thus additional scan time. According to the present embodiments, however, any low-resolution image of the body part may be used for training the machine learning model. Thus, instead of being limited to a scout reference image, as in the conventional SAMER technique, any other fully sampled prior scan or calibration measurement may be used by the trained machine learning model. The obtained motion parameters may be used to adapt the field of view during the acquisition of MRI dataset for prospective motion correction. Optionally, the guidance lines may be continued to be acquired in the original field of view without any adaptation. The pose parameters may in addition or alternatively be used in retrospective motion correction. For example, the method of the present embodiments is seamlessly compatible with an additional retrospective SAMER correction. Thus, an additional retrospective or even a purely retrospective application of the proposed trained machine learning model is possible.

The present embodiments use a fast trained machine learning model (e.g., neural network) that is applied to a dataset including the SAMER guidance line data. Since the present embodiments operate exclusively in k-space, there is no need for a prior reconstruction of data, making the method particularly fast. The obtained pose parameters may be readily used for prospective motion correction. This minimizes motion artifacts and second order artifacts, such as spin history artifacts, as well as k-space gaps, caused, for example, by rotational patient motion. A normal reconstruction (e.g., GRAPPA) may be used after the prospective correction, providing the motion corrected MR dataset almost immediately.

The method of the present embodiments may be executed on any medical or other MRI apparatus. The motion-corrected MR image is acquired from a field-of-view including a body part of a subject. The subject may be human or animal (e.g., a patient to be examined). The image dataset is, for example, acquired from a part of the body that is subject to undesired motion (e.g., the head or neck, a limb such as a leg, arm, knee, hand, or a part subjected to breathing motion such as the thorax or abdomen). The image dataset may be a three-dimensional (3D) dataset, acquired using two phase encoding directions, or the image dataset may be a two-dimensional (2D) image dataset, where a 2D image dataset includes one or, for example, a stack of 2D slices. A 2D image dataset is acquired using a slice selection gradient typically followed by phase encoding in one in-plane direction and frequency encoding in the other in-plane direction.

The imaging protocol may use any type of imaging sequence (e.g., a spin-echo or gradient echo sequence). The imaging sequence may, for example, be a TSE or TSE-type sequence (e.g., having T1-weighted, T2-weighted, or other contrast). The imaging sequence may be a non-steady-state sequence (e.g., one in which the signal intensity or contrast varies over an echo train; due to T1 and/or T2 relaxation, such as MPRAGE (Magnetization Prepared Rapid Gradient Echo Imaging) or TSE (Turbo Spin-Echo)). Further examples of the imaging protocol are a Sampling Perfection with Application optimized Contrast using different flip angle Evolution (SPACE) sequence, Susceptibility-Weighted Imaging (SWI), and Fluid-attenuated inversion recovery (FLAIR) sequence, but other types of imaging sequences are also possible, such as various gradient echo sequences.

The thereby acquired MR image dataset, also referred to as “image dataset,” “diagnostic image dataset,” or “high-resolution image,” may be reconstructed using retrospective motion correction techniques (e.g., the technique disclosed by D. Polak et al. in Magn. Reson. Med., vol. 87, pp. 163-178, 2022 and Magn. Reson. Med. 2023:1-14, doi: 10.1002/mrm.29534). The image dataset may be acquired for diagnostic purposes and thus may have a high spatial resolution of, for example, an in-plane resolution of 0.3 mm-3 mm or 0.4 mm-2 mm. The voxel size may be, for example, 0.5 to 12 mm³ or 2 to 8 mm³. For a 2D image, the in-plane resolution may, for example, be 0.3 mm-2 mm or 0.4 mm-1.2 mm.

The low-resolution image used in the method also covers the body part. The low-resolution image has an overlapping field-of-view to the MR image dataset, and the low-resolution image may have the same field-of-view, but not necessarily. In one embodiment, the low-resolution image does not have to be perfectly matched in terms of field-of-view and contrast to the guidance lines and MR image dataset. In case the image dataset is a stack of 2D slices, the low-resolution image may also include a stack of low-resolution 2D slices. However, it is possible that the low-resolution image includes fewer slices than the high-resolution image (e.g., only every second or third slice). The low-resolution image may have a spatial resolution of 2-8 mm or 3-5 mm (e.g., 4 mm) in the phase-encoding direction(s). In one embodiment, the acquisition of the low-resolution image is very rapid, requiring, for example, 1-5 s (e.g., 1-2 s). The low-resolution image may be acquired in the course of a calibration imaging scan, which may be acquired at the beginning of a patient examination (e.g. after the patient has been positioned in the MR scanner and before the diagnostic scans are performed). For example, the low-resolution image may be an imaging scan acquired to estimate the sensitivity profile of a multi-channel local coil that is used in that particular examination (PatRefScan), or a navigator or scout image used to plan the further diagnostic imaging protocols.

Thus, according to an embodiment, the low-resolution image has a different magnetic resonance contrast than the further sets of additional k-space lines, and, for example, is acquired in a calibration imaging scan performed before the imaging protocol.

The sets of additional k-space lines are acquired in a central region of k-space, where the central region may be covered by the low-resolution image. However, each set of additional k-space lines may include fewer k-space lines than would be required to reconstruct an image. For example, a set of k-space lines may include 1-8 or 1-4 k-space lines for each 2D slice of a 2D image dataset. In case the image dataset is a 3D image dataset, a set of k-space lines may include 2-32 or 3-8 k-space lines (e.g., 4 k-space lines). The set of additional k-space lines is acquired repeatedly during the imaging protocol (e.g., at at least approximately regular intervals). “At least approximately,” when used in this application, may be, for example, within ±15%, within ±10%, or within ±5%. The intervals at which the sets of additional k-space lines are acquired may be between 100 ms and 3000 ms or between 500 ms and 1500 ms. Thus, acts (b2) and (c) may be repeated at these intervals throughout the imaging protocol. Each set of additional k-space lines thereby provides information on the position of the subject at the point in time at which the set was acquired (e.g., one set of pose parameters). A motion trajectory of the subject during the imaging protocol is thereby obtained. The pose parameters may be rigid-body motion parameters, where each set of pose parameters includes, for example, three translational and three rotational parameters, or non-rigid pose parameters. “Additional” may be that the k-space lines are acquired in addition to those that are required under the imaging protocol to acquire the MR image (e.g., the additional k-space lines are redundant when it comes to image reconstruction). The sets of additional k-space lines may always be acquired at the same position in k-space at each acquisition, but this is not mandatory. Further, the sets of additional k-space lines may all have the same contrast, but that is not a requirement. It is a great advantage of using a trained machine learning model that pose parameters may also be extracted from sets of guidance lines having different contrasts (e.g., having been acquired at different TEs and/or at different positions during an echo train).

According to an embodiment, the imaging protocol includes a plurality of echo trains or “shots,” where one shot includes a plurality of magnetic resonance echoes (e.g., spin echoes and/or gradient echoes). During each echo, a k-space line is acquired. In most sequences, an echo train includes an excitation or preparation pulse, and then all echoes have their own excitation/refocusing pulses. There may be, for example, 1 to 1024 echoes or 8 to 256 echoes in one echo train. In 2D TSE sequences, an echo train may contain, for example, less than 40 or less than 30 echoes. In one embodiment, at least one set of additional k-space lines is acquired during at least some (e.g., more than 80%) of the echo trains.

According to an embodiment, the method further includes acquiring a reference set of additional k-space lines within a central region of k-space (e.g., at the beginning of the imaging protocol). The trained machine learning model is applied to a dataset including the low-resolution image in k-space notation, the reference set of additional k-space lines, and a further set of additional k-space lines. Thereby, the trained machine learning model is able to interpret the pose difference between the two sets of guidance lines. In one embodiment, the reference set of additional k-space lines is the same for each estimation of pose parameters during the imaging protocol.

By including a reference set of additional k-space lines into the dataset, to which the trained machine learning model is applied, the method becomes more robust. For example, the trained machine learning model may generate accurate pose parameters, even if the low-resolution image has a different contrast than the guidance lines. The reference set of guidance lines and the further set of guidance lines may have matching contrast. However, the trained machine learning model may even cope with contrast differences. Surprisingly, it has been found that the reference set of additional k-space lines needs not necessarily have been acquired particularly close in time to the lower resolution image. Nevertheless, the reference set of additional k-space lines may be acquired at the beginning of the imaging protocol. The low-resolution magnetic residence image may also be acquired before or at the beginning of the imaging protocol.

The pose parameters may be used for prospective and/or retrospective motion correction of the magnetic resonance image dataset, for notifying an operator or for triggering further actions.

According to an embodiment, the pose parameters are used to adapt a field-of-view of the magnetic resonance image dataset during the imaging protocol. In other words, the pose parameters are used for prospective motion correction. This may be done by comparing the latest set of pose parameters with the previous set, thereby finding the difference (e.g., a translation and/or a rotation), and translating and/or rotating the field-of-view of the imaging protocol accordingly. The new field-of-view is then used in the further acquisition of the imaging protocol (e.g., for the next shot).

According to an embodiment, the pose parameters are analyzed, and if the pose parameters indicate that subject motion has been above a certain threshold, the method includes an act of triggering alerting a user supervising during the image acquisition, triggering aborting or restarting the acquisition of the magnetic resonance image dataset, triggering re-acquiring the magnetic resonance data affected by the subject motion, or any combination thereof.

This is a feature that is only possible because the trained machine learning model is so fast that the pose parameters may be evaluated while the imaging protocol is being performed. Thereby, real-time feedback may be given to the radiologist (e.g., a warning may be issued). For example, the pose parameters may be further processed in order to predict whether aborting the imaging protocol is advantageous, or in order to trigger a re-acquisition of the most corrupted echo trains. Alternatively or additionally, the pose parameters may be used to display the detected motion trajectory.

According to an embodiment, the imaging protocol includes acquiring a plurality of echo trains (ETs), each echo train of the plurality of ETs including a plurality of echoes, where one k-space line is sampled during one echo, and where at least one further set of additional k-space lines is acquired in at least some of the echo trains (e.g., at the beginning of the echo train). The pose parameters estimated from a further set of additional k-space lines is used to adapt a field-of-view of the magnetic resonance image dataset in the same echo train in which the further set of additional k-space lines is acquired, or in the next echo train. This has the advantage that very fast prospective motion correction is possible. Since an echo train may have a duration of, for example, between 100 ms and 3000 ms or between 500 ms and 1500 ms, correcting the field-of-view after each echo train already gives a very good motion correction. However, the method of the present embodiments is so fast that an adaptation of the field-of-view is possible even during one echo train (e.g., 1 to 20 or 1 to 5 times per echo train). Thereby, patient motion may be corrected at a time resolution of, for example, between 30 ms and 200 ms or between 50 ms and 150 ms.

In other words, the trained machine learning model allows to increase the number of GL sets per ET. For example, a set of GLs may be acquired at the beginning, middle, and end of an ET. Alternatively, one GL set may be acquired before every echo. Thereby, the temporal resolution may be increased. In a further embodiment, the GLs may be positioned at any different time point instead of at the standard ˜TR/2 time point during the sequence run (e.g., during contrast waiting times). In SAMER, for each position of GL during the echo train, a separate scout is to be acquired. This is not required for the method of the present embodiments, provided the network is trained accordingly. In a specific implementation, the NN may use GLs at the beginning of each ET or at any time before the ET for fast detection and prospective correction of the ET, thereby closing k-space gaps and accounting for spin history effects.

According to an embodiment, the magnetic resonance image dataset is acquired using a multi-channel coil array. The low-resolution image in k-space notation and the further set of additional k-space lines are pre-processed by removing a peripheral region of k-space and/or by reducing a number of channels. Thereby, the dataset that is input into the trained machine learning model is cropped to a smaller size, making the processing faster.

According to an embodiment, the trained machine learning model includes a neural network. According to an embodiment, the trained machine learning model includes a convolutional neural network (CNN) (e.g., a DenseNet). A DenseNet is a type of convolutional neural network that utilizes dense connections between layers, through dense blocks, where all layers having matching feature-map sizes are connected directly with each other. Thereby, a fast NN may be realized. The NN may, for example, utilize 3D complex convolutions with dilation of 8. The NN may include 2 to 4 dense blocks, each dense block followed by a transition layer. The transition layer reduces the number of channels and may in the first and second layer also reduce the spatial size (e.g., by 2×2×2).

Other deep learning architectures are also possible. For example, the trained machine learning model may include a transformer-based network.

The NN operates exclusively in k-space. The input to the NN may be a dataset including three components, each in k-space notation: 1. a reference set of guidance lines, which is, for example, the first set of guidance lines acquired during the imaging protocol (e.g., acquired during the first echo train); 2. the set of GL from which the pose parameters are to be extracted; and 3. a low-resolution image (e.g. a calibration or reference scan).

Before applying the NN, the k-space data may be pre-processed (e.g., to reduce the amount of data and thus to make the NN faster). For example, the GLs and the low-resolution image may be coil-compressed, where the number of channels is reduced (e.g., from 16 to 4). Further, the k-space data may be cropped to cover only a central portion of k-space. Thereby, the dataset may achieve a size of, for example, 32×16×16×4×3×2, where the dimensions are kₓ×k_{y}×k_{z}×number of coil channels×number of components (reference GL, GL, and low-resolution image)×2 (complex numbers). The output of the NN may be a set of rigid motion pose parameters (e.g., three translational and three rotational parameters).

According to an embodiment, the pose parameters are used for retrospective motion correction of the magnetic resonance image dataset. Thereby, the pose parameters are used in the image reconstruction, which may use a forward model incorporating motion parameters, as described below. Alternatively, a second machine learning model (e.g., a second NN) may be trained to reconstruct the diagnostic image from the k-space data and the motion trajectory obtained from the GLs.

When using the pose parameters for retrospective motion correction, a method for generating a motion-corrected magnetic resonance image dataset of an object, for example, is carried out. The method includes receiving k-space data acquired using the acquisition method according to an embodiment, receiving estimated pose parameters for each further set of additional k-space lines, and estimating the motion-corrected magnetic resonance image dataset by minimizing the data consistency error between the k-space data acquired in the imaging protocol and a forward model described by an encoding matrix. The encoding matrix includes the pose parameters for each further set of additional k-space lines, Fourier encoding, and optionally subsampling and/or coil sensitivities of a multi-channel coil array. Thereby, retrospective motion correction is possible.

The pose parameters are provided by the trained machine learning model of the method of the present embodiments. The motion-corrected image dataset may then be estimated by minimizing the data consistency error between the k-space data acquired in the imaging protocol and a forward model. The forward model may be a “SENSE+motion” model, as described in K. P. Pruessmann, M. Weiger, M. B. Scheidegger, and P. Boesiger, “SENSE: sensitivity encoding for fast MRI,” Magn. Reson. Med., vol. 42, no. 5, pp. 952-962, 1999.

In an example, the mathematical model used is an extension of SENSE parallel imaging, with rigid-body motion parameters included into the forward model. The forward model or encoding operator E_{θ} for a given patient motion vector θ (e.g., including pose parameters over time) relates the motion-free image x to the acquired multi-channel k-space data s. At each time point i that is considered (e.g., the acquisition time of the sets of guidance lines), the subject's position is described by a new set of six rigid-body motion parameters θᵢ that describe the 3D position of the object. Accordingly, the multi-channel k-space data sᵢ acquired at time point i may be related to the 3D image volume x through image rotations Rᵢ, image translations Tᵢ, coil sensitivity maps C, Fourier operator F, and under-sampling mask Mᵢ by the following formula 1:

$${s}_{i} = {E}_{{θ}_{i}} x = {M}_{i} F C {T}_{{θ}_{i}} {R}_{{θ}_{i}} x \qquad [ 1 ]$$

In prior art methods, both the motion corrected image vector x and the motion trajectory θ are estimated by performing an alternating, repeated optimization between the image vector (formula 2) and the motion vector (formula 3):

$$[ \hat{x} ] = arg {min}_{x} {‖ {E}_{\hat{θ}} x - s ‖}_{2} \qquad [ 2 ]$$

$$[ \hat{θ} ] = arg {min}_{θ} {‖ {E}_{θ} \hat{x} - s ‖}_{2} \qquad [ 3 ]$$

This may lead to convergence issues, as updates of x and θ will be computed on inaccurate information. Further, the reconstruction is computationally demanding, as repeated updates of x (e.g., millions of imaging voxels) are needed.

In the method of the present embodiments, the motion trajectory θ is estimated from the sets of guidance lines and a trained machine learning model, so that equation [3] does not have to be solved. For the final image reconstruction, the pose parameters from each set of guidance lines are combined, and the motion-mitigated image is obtained from solving only the standard least-squares problem of equation [2].

According to a further aspect of the present embodiments, a computer-implemented method for providing a trained machine learning model is provided. The method includes receiving input training data including a low-resolution magnetic resonance image of a body part in k-space notation and a set of additional k-space lines from a central region of k-space. The low resolution image has been derived from an example magnetic resonance image, and the set of additional k-space lines has been derived from the same example magnetic resonance image after the image has been rotated and/or translated by a set of pose parameters. The method also includes: receiving output training data including the set of pose parameters; training a machine learning model based on the input training data and the output training data; and providing the trained machine learning model. According to an embodiment, the input training data is derived from previously acquired example MR images (e.g., clinical 3D image datasets) that may be images of the same body part or different body parts from various subjects. These image datasets are optionally modified (e.g., flipped and/or stretched) for optional data augmentation and then shifted and rotated to simulate patient motion, using pre-determined motion trajectories, and Fourier-transformed back into k-space, where the guidance lines are extracted for each pose during the motion trajectory. A low-resolution image is also simulated from the example MR image. Optionally, for data augmentation, the example magnetic resonance image may be derived from a first acquired MR image by image modifications. In an embodiment, such modifications may include one or all of the following acts: image flips, mirroring of the image, and stretching. Optionally, real measured motion cases may enter the training when the respective motion trajectory is determined by a reference method. By augmenting the data, a low number of, for example, 10 to 50 3D acquired MR images may be sufficient to train the machine learning model so that the machine learning model correctly predicts the pose parameters from the guidance lines and the low-resolution image. According to an embodiment, a set of reference guidance lines is also used as input training data.

According to a further aspect of the present embodiments, a computer program is provided that includes program code that causes a magnetic resonance imaging apparatus (e.g., the apparatus described herein) to execute the method of the present embodiments (e.g., the method of the present embodiments for acquiring an MR image dataset). However, the program code may also encode the described training method, and the program code may run on a computer as described herein.

According to a further aspect, the present embodiments are directed to a non-transitory computer-readable medium containing a computer program as described herein. The computer-readable medium may be any digital storage medium, such as a hard disc, a cloud, an optical medium such as a CD or DVD, a memory card such as a compact flash, memory stick, a USB-stick, multimedia stick, secure digital memory card (SD), etc.

In a further aspect of the present embodiments, a magnetic resonance imaging apparatus that includes a radio frequency controller configured to drive an RF-coil (e.g., including a multi-channel coil-array, a gradient controller configured to control gradient coils, and a control unit configured to control the radio frequency controller and the gradient controller to execute the imaging protocol according to the present embodiments) is provided. The MRI apparatus further includes a processing unit configured to apply a trained machine learning model to a dataset including a low-resolution image in k-space notation and a set of additional k-space lines, where a set of pose parameters is generated. The MRI-apparatus may be a commercially available MRI-apparatus that has been programmed to perform the method of the present embodiments. The processing unit may be part of the control unit, or the processing unit may be separate. The processing unit may, for example, be any CPU or GPU. The processing unit may be part of a computer, where the computer may be a PC, a server, a console of an MRI apparatus, but the processing unit may also be a computer that is remote from the MRI apparatus. The computer may be connected with the MRI apparatus through the internet. Accordingly, the computer may also be a cloud computer, a remote server, etc. The computer may also be a mobile device, such as a laptop, tablet computer, or mobile phone. The present embodiments are also directed to such a processing unit (e.g., a processing unit configured to apply a trained machine learning model to a dataset including a low-resolution image in k-space notation and a set of additional k-space lines, where a set of pose parameters is generated).

All features disclosed with regard to the acquisition method may be combined with all features of the training method and vice versa. Also, all features of the disclosed methods may be embodied in the MRI apparatus, computer program, and computer-readable storage medium (e.g., non-transitory computer-readable storage medium) according to other aspects of the present embodiments and vice versa.

### BRIEF DESCRIPTION OF THE DRAWINGS

The accompanying drawings illustrate various example embodiments of various aspects of the invention. In the drawings:

FIG. 1 is a schematic representation of a magnetic resonance imaging (MRI) apparatus according to an embodiment;

FIG. 2 is a schematic representation of a three-dimensional k-space;

FIG. 3 is a flow diagram illustrating a method according to an embodiment;

FIG. 4 is a schematic diagram illustrating an NN architecture;

FIG. 5 is a schematic timeline of an imaging protocol illustrating embeddings of GLs for retrospective motion correction according to a first embodiment;

FIG. 6 is a schematic timeline of an imaging protocol illustrating embeddings of the GLs for retrospective motion correction according to a second embodiment;

FIG. 7 is a schematic timeline of an imaging protocol illustrating embeddings of the GLs for prospective motion correction according to a first embodiment;

FIG. 8 is a schematic timeline of an imaging protocol illustrating embeddings of the GLs for prospective motion correction according to a second embodiment;

FIG. 9 is a schematic timeline of an imaging protocol illustrating embeddings of the GLs for prospective and retrospective motion correction according to a first embodiment.

Similar elements are designated with the same reference signs in the drawings.

### DETAILED DESCRIPTION

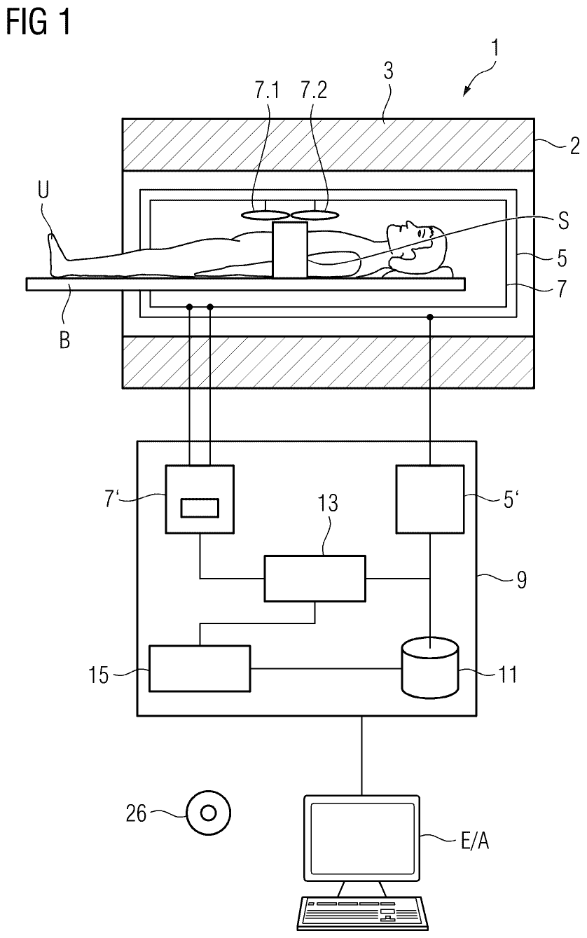

FIG. 1 schematically shows a magnetic resonance (MR) apparatus 1. The MR apparatus 1 has an MR data acquisition scanner 2 with a magnet 3 that generates a constant magnetic field, a gradient coil arrangement 5 that generates gradient fields, one or more radiofrequency (RF) antennas 7 (e.g., several RF antennas 7) for radiating and receiving RF signals, and a control computer 9 configured to perform a method of the present embodiments. The radio-frequency antennas 7 may include a multi-channel coil array including at least two coils (e.g., the schematically shown coils 7.1 and 7.2) that may be configured to transmit and/or receive RF signals (MR signals).

In order to acquire MR data from a 3D volume S of an examination subject U (e.g., a patient or a phantom), the subject U is introduced on a bed B into a measurement volume of the scanner 2. MR data may be acquired using a method according to an embodiment of from the 3D volume S. The control computer 9 controls the MR apparatus 1, and the gradient coil arrangement 5 with a gradient controller 5′ and the RF antenna 7 with a RF transmit/receive controller 7′. The RF antenna 7 has multiple channels corresponding to the multiple coils 7.1, 7.2 of the coil arrays, in which signals may be transmitted or received. The control computer 9 also has an imaging protocol processor 15 that determines the imaging protocol. A control unit 13 of the control computer 9 is configured to execute all the controls and computation operations required for acquisitions. The control unit may also perform processing steps (e.g., applying the trained machine learning model). Acquired data or data determined in the process may be stored in a memory 11 of the control computer 9. A user may enter control commands and/or view displayed results (e.g., image data) via an input/output interface E/A. A non-transitory data storage medium 26 may be loaded into the control computer 9 and may be encoded with programming instructions (e.g., program code) that cause the control computer 9, and the various functional units thereof described above, to implement any or all embodiments of the method according to the present embodiments.

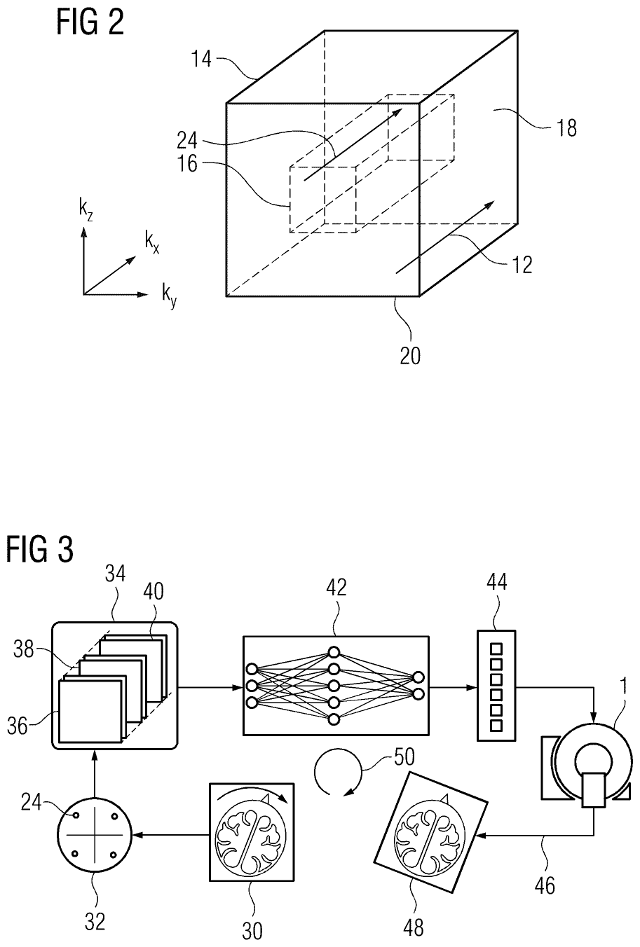

FIG. 2 illustrates a three-dimensional k-space 14 having dimension kₓ in frequency encoding (e.g., readout) direction, and k_{y} and k_{z} in the phase encode plane 20. A k-space line acquired during one echo is illustrated at 12. The k-space volume 14 includes a central region 16 and a periphery 18. The additional k-space lines 24 as well as the k-space lines acquired for the low-resolution scout are located in a central region 16. In the phase encode plane 20, the central region 16 may cover about 1/12 to 1/16 in each direction (e.g., less than 1/100 of the total square phase encode plane 20).

FIG. 3 illustrates a method according to an embodiment. The illustrated acts are carried out a number of times during an imaging protocol, during which a diagnostic image is to be acquired, as indicated by arrow 50. Starting at 30, the patient might rotate his head, which is the body part of which an image is taken, during the imaging protocol. Directly after the motion, a set of guidance lines 32 is acquired. The picture at 32 shows the phase encode plane and four guidance lines 24, one in each quadrant. The data from the set of guidance lines 32 is copied a number of times into each quadrant of a component of the input dataset for the NN, as illustrated at 34. The input dataset 34 to the NN includes the guidance lines 36 that have just been acquired at 32, a set of reference GLs 38 that have been acquired some time earlier during the imaging protocol, and the low-resolution image dataset 40 (e.g., a calibration scan). The NN illustrated at 42 is applied to the input dataset 34, and the output is a set of pose parameters 44. These pose parameters are used by the MRI apparatus 1 to adjust the field-of-view (e.g., by updating the gradient and frequency settings). Because the NN requires so little processing time, this may be done practically in real time after acquiring the GLs 24. Thus, the imaging protocol will continue with an updated field-of-view, as illustrated at 48. This sequence may be carried out once or even a number of times during one echo time.

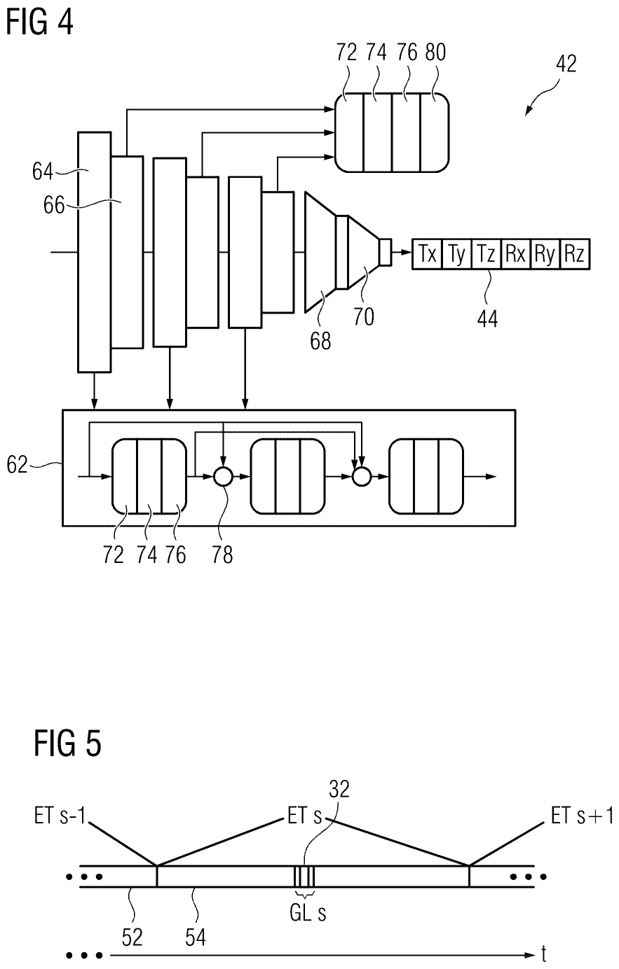

FIG. 4 illustrates an example NN 42 that may be used as a trained machine learning model. The NN 42 may include 2 to 4 dense blocks 64, each dense block followed by a transition layer 66. The dense blocks 64 may utilize 3D complex convolutions with dilation of 8. The transition layer 66 reduces the number of channels and may in the first and second layer also reduce the spatial size by 2×2×2. A dense block is further illustrated at 62, showing that each dense block has a batch normalization layer 72, followed by an activation function 74, followed by a complex convolution 76. Concatenation is illustrated at 78. The transition layers 66 include a batch normalization 72, followed by an activation function 74, followed by a complex convolution 76, followed by max. pooling 80. In this example, the first DenseBlock and last transition layer use tanh activation, and the rest uses leaky ReLU. The last block of the NN maps the feature vector to the pose parameters 44 in two linear layers 68, 80 with tanh activation.

FIGS. 5 to 9 shows sketches of different example embeddings of the GLs and NN for a retrospective, prospective, and combined motion correction. The horizontal bars 52 indicate the timeline of the imaging protocol. The bars 52 are divided into echo trains (ETs) 54. The position of each GL set (e.g., in this example, each set includes 4 additional k-space lines) is indicated by four vertical bars 32. The number of lines within one set and the number of GL sets acquired, as well as the number of GL sets used for prospective and retrospective correction, however, are flexible and any combination is possible according to the present embodiments. A shading indicates a prospective adaption of the field-of-view to the motion estimate of the NN. A retrospective estimation may also correct only parts of the ET and/or parts of neighboring ETs.

FIG. 5 illustrates a case where the pose parameters derived from the GLs are used for retrospective motion correction only. The set of GLs 32 is acquired in the middle of an ET 54, and the diagnostic image dataset is reconstructed using the pose parameters and a motion model after the acquisition.

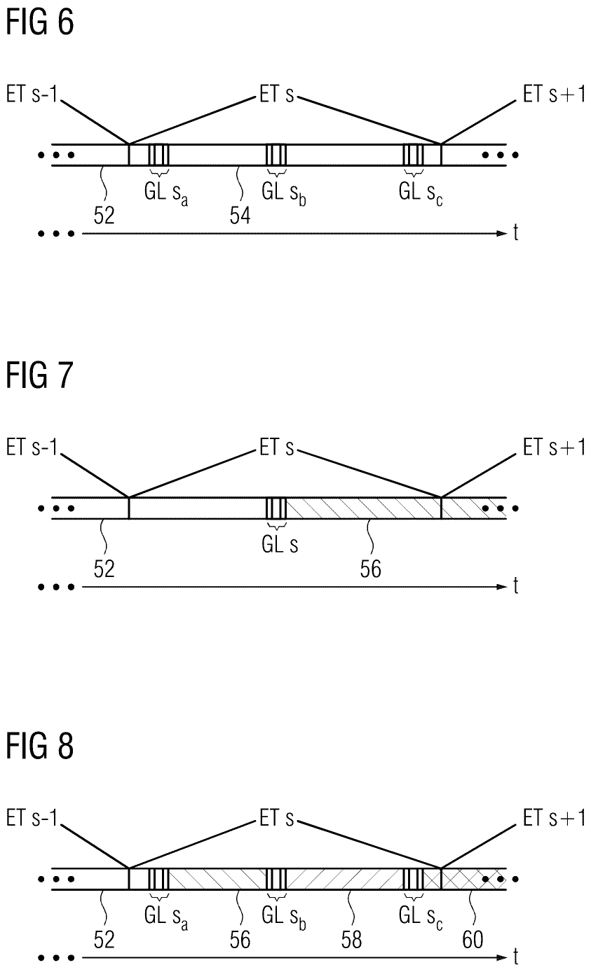

FIG. 6 illustrates retrospective motion correction using multiple (e.g., three) GL sets 32 per echo train 54. Motion parameters are estimated for every GL set 32, and the diagnostic image dataset is reconstructed using the pose parameters and a motion model after the acquisition.

FIG. 7 illustrates prospective motion correction with a single GL set 32 acquired per echo train. After the acquisition of each GL set 32, pose parameters are estimated, and the field-of-view is adapted to this pose directly afterwards, in the shaded area 56.

FIG. 8 illustrates prospective motion correction with multiple GL sets 32 acquired per echo train. After the acquisition of each GL set 32, pose parameters are estimated, and the field-of-view is adapted to this pose in the shaded areas 56, 58, 60.

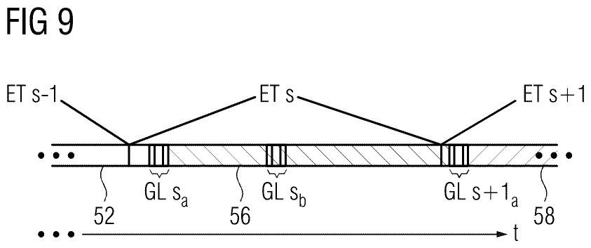

FIG. 9 illustrates prospective and retrospective motion correction with multiple GL sets 32 acquired per echo train. GL set sₐ is acquired in echo train s, and GL set s+1ₐ is acquired in echo train s+1. After the acquisition of GL sets sₐ and s+1ₐ, which are acquired at the beginning of their respective echo trains, pose parameters are estimated, and the field-of-view is adapted to this pose for the remaining ET in the shaded areas 56, 58. Residual artifacts may be corrected retrospectively using the GL sets se acquired in the middle of each echo train.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for acquiring a magnetic resonance image dataset of a body part of a subject, the method comprising:
acquiring the magnetic resonance image dataset using an imaging protocol, wherein spatial encoding is performed using phase encoding gradients along at least one phase encoding direction, and frequency encoding gradients along a frequency encoding direction, and wherein k-space is sampled during the imaging protocol by acquiring a plurality of imaging k-space lines oriented along the frequency encoding direction, and having different positions in the at least one phase encoding direction,
acquiring or providing a low-resolution magnetic resonance image of the body part;
acquiring further sets of additional k-space lines within a central region of k-space at intervals throughout the imaging protocol; and
applying a trained machine learning model to a dataset comprising the low-resolution image in k-space notation and a further set of additional k-space lines,
wherein a set of pose parameters is generated, the pose parameters being an estimation of the pose of the subject during the acquisition time of the further set of additional k-space lines.

2. The method of claim 1, further comprising acquiring a reference set of additional k-space lines within a central region of k-space.

3. The method of claim 2, wherein acquiring the reference set of additional k-space lines within the central region of k-space comprises acquiring the reference set of additional k-space lines within the central region of k-space at a beginning of the imaging protocol.

4. The method of claim 1, wherein the pose parameters are used for prospective, retrospective, or prospective and retrospective motion correction of the magnetic resonance image dataset, for notifying an operator, for triggering further actions, or for notifying the operator and for triggering the further actions.

5. The method of claim 1, further comprising:
acquiring a reference set of additional k-space lines within a central region of k-space,
wherein the trained machine learning model is applied to a dataset comprising the low-resolution image in k-space notation, the reference set of additional k-space lines, and a further set of additional k-space lines.

6. The method of claim 1, wherein the pose parameters are used to adapt a field-of-view of the magnetic resonance image dataset during the imaging protocol.

7. The method of claim 1, wherein the pose parameters are used for retrospective motion correction of the magnetic resonance image dataset.

8. The method of claim 1, wherein the pose parameters are further processed and, when the pose parameters indicate that subject motion has been above a certain threshold, the method further comprises triggering alerting a user supervising during the image acquisition, aborting or restarting the acquisition of the magnetic resonance image dataset, re-acquiring the magnetic resonance data affected by the subject motion, or any combination thereof.

9. The method of claim 1, wherein the low-resolution image has a different magnetic resonance contrast than the further sets of additional k-space lines, and is acquired in a calibration imaging scan performed before the imaging protocol.

10. The method of claim 1, wherein the imaging protocol comprises acquiring a plurality of echo trains, each echo train of the plurality of echo trains comprising a plurality of echoes, wherein one k-space line is sampled during one echo, and
wherein at least one further set of additional k-space lines is acquired in at least some echo trains of the plurality of echo trains, preferably at the beginning of the echo train, and wherein the pose parameters (44) estimated from a further set of additional k-space lines is used to adapt a field-of-view of the magnetic resonance image dataset in the same echo train (54) in which the further set of additional k-space lines is acquired, or in the next echo train.

11. The method of claim 10, wherein the at least one further set of additional k-space lines is acquired in the at least some echo trains at the beginning of the echo train.

12. The method of claim 1, wherein the magnetic resonance image dataset is acquired using a multi-channel coil array, and
wherein the low-resolution image in k-space notation and the further set of additional k-space lines are pre-processed by removing a peripheral region of k-space, by reducing a number of channels, or by removing the peripheral region of k-space and by reducing the number of channels.

13. The method of claim 1, wherein the trained machine learning model comprises a convolutional neural network.

14. The method of claim 13, wherein the convolutional neural network is a DenseNet.

15. A method for generating a motion-corrected magnetic resonance image dataset of an object, the method comprising:
receiving k-space data acquired using an acquisition method, the acquisition method comprising:
acquiring a magnetic resonance image dataset using an imaging protocol, wherein spatial encoding is performed using phase encoding gradients along at least one phase encoding direction, and frequency encoding gradients along a frequency encoding direction, and wherein k-space is sampled during the imaging protocol by acquiring a plurality of imaging k-space lines oriented along the frequency encoding direction, and having different positions in the at least one phase encoding direction,
acquiring or providing a low-resolution magnetic resonance image of a body part;
acquiring further sets of additional k-space lines within a central region of k-space at intervals throughout the imaging protocol; and
applying a trained machine learning model to a dataset comprising the low-resolution image in k-space notation and a further set of additional k-space lines, wherein a set of pose parameters is generated, the pose parameters being an estimation of the pose of the subject during the acquisition time of the further set of additional k-space lines;
receiving estimated pose parameters for each further set of additional k-space lines; and
estimating the motion-corrected magnetic resonance image dataset, the estimating of the motion-corrected magnetic resonance image dataset comprising minimizing the data consistency error between the k-space data acquired in the imaging protocol and a forward model described by an encoding matrix,
wherein the encoding matrix includes the pose parameters for each further set of additional k-space lines and Fourier encoding.

16. The method of claim 15, wherein the encoding matrix further includes subsampling, coil sensitivities of a multi-channel coil array, or a combination thereof.

17. A computer-implemented method for providing a trained machine learning model, the computer-implemented method comprising:
receiving input training data comprising a low-resolution magnetic resonance image of a body part in k-space notation and a set of additional k-space lines from a central region of k-space, wherein the low resolution image has been derived from an example magnetic resonance image, and the set of additional k-space lines has been derived from the same example magnetic resonance image after the image has been rotated, translated, or rotated and translated by a set of pose parameters;
receiving output training data comprising the set of pose parameters;
training a machine learning model based on the input training data and the output training data; and
providing the trained machine learning model.

18. A magnetic resonance imaging apparatus comprising:
a processor configured to acquire a magnetic resonance image dataset of a body part of a subject, the acquisition comprising:
acquisition of the magnetic resonance image dataset using an imaging protocol, wherein spatial encoding is performed using phase encoding gradients along at least one phase encoding direction, and frequency encoding gradients along a frequency encoding direction, and wherein k-space is sampled during the imaging protocol by acquiring a plurality of imaging k-space lines oriented along the frequency encoding direction, and having different positions in the at least one phase encoding direction,
acquisition or provision of a low-resolution magnetic resonance image of the body part;
acquisition of further sets of additional k-space lines within a central region of k-space at intervals throughout the imaging protocol; and
application of a trained machine learning model to a dataset comprising the low-resolution image in k-space notation and a further set of additional k-space lines, wherein a set of pose parameters is generated, the pose parameters being an estimation of the pose of the subject during the acquisition time of the further set of additional k-space lines;
a radio frequency (RF) controller configured to drive an RF-coil comprising a multi-channel coil array;
a gradient controller configured to control gradient coils; and
a control unit configured to control the radio frequency controller and the gradient controller to execute the imaging protocol.
